(19) United States
(12) Patent Application Publication
(10) Pub. No.: US 2025/0222158 A1
(43) Pub. Date: Jul. 10, 2025

(54) An Apparatus for Disinfecting a Target Surface or Air

(71) Applicant: Germicidal Technology SDN BHD, Iskandar Puteri, Johor (MY)

(72) Inventor: Ah Eng SIAW, Johor Bahru, Johor (MY)

(73) Assignee: Germicidal Technology SDN BHD, Iskandar Puteri, Johor (MY)

(21) Appl. No.: 18/862,022

(22) PCT Filed: Apr. 8, 2022
(86) PCT No.: PCT/MY2022/050022

§ 371 Date: Oct. 31, 2024

(51) Int. Cl. A61L 9/18; A61L 2/08

(52) U.S. Cl. CPC A61L 9/18; A61L 2/084; A61L 2202/11; A61L 2209/12

(57) ABSTRACT

The present invention relates to the field of disinfection technology. More particularly, the present invention relates to an apparatus for disinfecting a target surface or air comprising (a) a light-emitting device; and (b) a coating deposited onto a surface of the light-emitting device, wherein the coating is derived from a composition comprising vibration-energy-containing nanoparticles, a binder, an acid-based surface additive, and an organic solvent, whereby the apparatus illuminates the target surface or air upon use thereby transferring the vibration energy of the vibration-energy-containing nanoparticles thereonto so as to disinfect the target surface or air.

### FIELD OF INVENTION

The present invention relates to the field of disinfection technology. More particularly, the present invention relates to an apparatus for disinfecting a target surface or air.

### BACKGROUND OF THE INVENTION

Infectious diseases can be transmitted from contaminated surfaces or space via physical contact or airborne transmission. This can be prevented by cleaning or disinfecting the contaminated surfaces or space. Cleaning involves the use of soap or detergent, whereas disinfection involves the use of a product or process to inactivate microbes responsible for the infectious diseases. Disinfection technology is vital in preventing and controlling the spread of infectious diseases such as COVID-19 caused by SARS-CoV-2 virus.

Conventionally, ultraviolet (UV) irradiation is used for disinfection. In particular, the UV irradiation inactivates microbes by destroying their nucleic acids and disrupting their DNA, thereby leaving the microbes unable to perform cellular functions. For example, US Patent No. U.S. Pat. No. 11,040,121B2 discloses a portable UV device for disinfection. The portable UV device comprises a germicidal UV light source disposed within a housing comprising a motorized unit, a hydraulic system, and an actuator. When not in use, the germicidal UV light source resides within the housing. When in use, the germicidal UV light source is released from the housing in a vertical and rotational manner. However, UV irradiation technology for surface disinfection can be costly due to the use of UV light bulb. Additionally, UV irradiation can damage structures of human eyes. Side effects are such as corneal damage, cataracts, and macular degeneration. Alternatively, Hwang et al. (doi: 10.1038/s41467-020-15004-6) discloses photobactericidal polymer containing crystal and thiolated gold nanocluster for killing microbes on a polymer upon activated by a light source. However, this technology requires the polymer to be in direct contact with the microbes, thereby rendering it inconvenient for use.

Therefore, it is desirable to provide a disinfection technology that is affordable and convenient to use while not inducing the aforementioned side effects. The present invention provides a solution to the problems.

### SUMMARY OF INVENTION

One aspect of the present invention is to provide a disinfection apparatus that is affordable and convenient to use while not inducing the side effects such as corneal damage, cataracts, and macular degeneration.

Another aspect of the present invention is to provide a readily available disinfecting apparatus in which common light emitting device can be modified thereinto. For instance, the light-emitting device is a portable handheld electrical device such as torch light and mobile phone or a non-portable electrical device such as streetlight and wall lamp. The light can be provided by light emitting diode (LED), fluorescent light bulb, and incandescent light bulb.

More particularly, the present invention aims to provide an apparatus for disinfecting a target surface or air comprising a light-emitting device and a coating deposited thereonto. The coating comprises vibration-energy-containing nanoparticles in which atoms of the nanoparticles are in a state of energy excitation that vibrate for a period of time upon being subjected to a vibration force. When in use, the apparatus illuminates the target surface or air thereby transferring the vibration energy of the vibration-energy-containing nanoparticles thereonto so as to disinfect the target surface or air.

At least one of the preceding aspects is met, in whole or in part, in which the embodiment of the present invention describes an apparatus for disinfecting a target surface or air comprising (a) a light-emitting device; and (b) a coating deposited onto a surface of the light-emitting device, wherein the coating is derived from a composition comprising vibration-energy-containing nanoparticles, a binder, an acid-based surface additive, and an organic solvent, whereby the apparatus illuminates the target surface or air upon use thereby transferring the vibration energy of the vibration-energy-containing nanoparticles thereonto so as to disinfect the target surface or air.

In a preferred embodiment of the present invention, the vibration-energy-containing nanoparticles are present at about 0.1% to about 10% by weight of the composition, the binder is present at about 0.1% to about 30% by weight of the composition, the surface additive is present at about 0.1% to about 8% by weight of the composition, and the organic solvent is present at about 75% to about 94% by weight of the composition.

In the preferred embodiment of the present invention, the vibration-energy-containing nanoparticles are in a state of energy excitation that vibrate at a frequency of 1 to 1000 kHz for a predetermined period upon being subjected to a vibration force at the frequency for at least 6 hours.

Preferably, the vibration-energy-containing nanoparticles are metal-based nanoparticles, rare earth-based nanoparticles, graphene nanoparticles, or any combinations thereof.

More preferably, the metal-based nanoparticles are derived from metal, metal oxide, metal nitrate, metal sulfate, or any combinations thereof.

More preferably, the rare earth-based nanoparticles are derived from scandium, yttrium, lanthanum, cerium, praseodymium, neodymium, promethium, samarium, europium, gadolinium, terbium, dysprosium, holmium, erbium, thulium, ytterbium, lutetium, or any combinations thereof.

Preferably, the binder is a silane.

Preferably, the acid-based surface additive is sulphuric acid, phosphoric acid, nitric acid, citric acid, or hydrochloric acid.

Further to the preferred embodiment of the present invention, the coating has a thickness of about 1 μm to about 10 μm.

### DETAILED DESCRIPTION OF THE INVENTION

Hereinafter, the present invention shall be described according to the preferred embodiments of the present invention and by referring to the accompanying description. However, it is to be understood that limiting the description to the preferred embodiments of the present invention is merely to facilitate discussion of the present invention and it is envisioned that those skilled in the art may devise various modifications without departing from the scope of the appended claims.

The present invention is an apparatus for disinfecting a target surface or air. Preferably, the apparatus comprises a light-emitting device and a coating deposited onto a surface of the light-emitting device. Preferably, the light-emitting device is a portable handheld electrical device such as torch light and mobile phone or a non-portable electrical device such as streetlight and wall lamp. The light can be provided by light emitting diode (LED), fluorescent light bulb, or incandescent light bulb. The light-emitting device can be powered by a built-in power source (such as a battery) and/or an external power source (with connections to existing power outlets). Wherever necessary, an existing light-emitting device can be converted into the disinfecting apparatus by coating the aforementioned composition thereon.

According to a preferred embodiment of the present invention, the coating is derived from a composition comprising vibration-energy-containing nanoparticles, a binder, an acid-based surface additive, and an organic solvent, whereby the apparatus illuminates the target surface or air upon use thereby transferring the vibration energy of the vibration-energy-containing nanoparticles thereonto so as to disinfect the target surface 10 or air. Preferably, the vibration-energy-containing nanoparticles are present at about 0.1% to about 10% by weight of the composition, the binder is present at about 0.1% to about 30% by weight of the composition, the surface additive is present at about 0.1% to about 8% by weight of the composition, and the organic solvent is present at about 75% to about 94% by weight of the composition. A lower amount of vibration-energy-containing nanoparticles is not preferable as it may not provide sufficient vibration for disinfection when in use, whereas a higher amount of vibration-energy-containing nanoparticles does not provide notable improvement.

According to the preferred embodiment of the present invention, the vibration-energy-containing nanoparticles are in a state of energy excitation that vibrate at a frequency of 1 to 1000 kHz for a predetermined period upon being subjected to a vibration force. The vibration force is preferably provided for a sufficiently long period of time to ensure that atoms of the nanoparticles capture and hold the energy from the vibration force for the predetermined period of time. Preferably, the vibration force is provided for at least 6 hours. During the process, the atoms are excited to vibrate vigorously for a period of time at a frequency similar to the frequency of the vibration force. By way of example, the vibration force can be provided by means of ultrasonication. In some embodiments of the present invention, the vibration-energy-containing nanoparticles can hold vibration energy and vibrate for 6 months.

Preferably, the vibration-energy-containing nanoparticles are metal-based nanoparticles, rare earth-based nanoparticles, graphene nanoparticles, or any combinations thereof. More preferably, the metal-based nanoparticles are derived from metal, metal oxide, metal nitrate, metal sulfate, or any combinations thereof. For example, the metal-based nanoparticles are colloidal copper, platinum oxide, silver oxide, titanium oxide, tin oxide, gold oxide, silver nitrate, silver citrate, copper sulfate, or a combination thereof. On the other hand, the rare earth-based nanoparticles are preferably derived from scandium, yttrium, lanthanum, cerium, praseodymium, neodymium, promethium, samarium, europium, gadolinium, terbium, dysprosium, holmium, erbium, thulium, ytterbium, lutetium, or any combinations thereof.

Yet, according to the preferred embodiment of the present invention, the organic solvent serves as a medium for distributing the vibration-energy-containing nanoparticles onto the surface of the light-emitting device during deposition of the coating. Preferably, the organic solvent used herein does not react with the vibration-energy-containing nanoparticles so that it does not alter the vibration energy stored in the vibration-energy-containing nanoparticles. Preferably, the organic solvent is an alcohol, a silicone oil, or a combination thereof. More preferably, the alcohol is methanol, ethanol, propanol, or any combinations thereof, whereas the silicone oil is hexamethyldisiloxane, octamethyltrisiloxane, decamethylcyclopentasiloxane, polydimethylsiloxane, octamethylcyclotetrasiloxane, or any combinations thereof. Advantageously, the silicone oil provides a smooth appearance and anti-stick characteristics to the coating to prevent adherence of solid impurities such as dusts.

Still, according to the preferred embodiment of the present invention, the binder is used to enhance binding of the vibration-energy-containing nanoparticles onto the surface of the light-emitting device during the deposition. Additionally, the binding allows the composition to be cured at room temperature to form the coating. Preferably, the binder is a silane.

Further to the preferred embodiment of the present invention, the acid-based surface additive is an acid which enhances binding of the vibration-energy-containing nanoparticles onto the surface of the light-emitting device. In particular, the acid lowers pH of the composition, thereby promoting etching of the vibration-energy-containing nanoparticles onto the surface of the light-emitting device by forming bonds therebetween. Preferably, the pH of the composition is adjusted to about 5 to about 6 for the aforementioned purpose. A pH lower than 5 is not preferable as it may cause corrosion to the surface of the light-emitting device. On the other hand, an alkaline composition having a pH of 8 or higher is not desirable as the coating derived therefrom may detach from the surface of the light-emitting device. Preferably, the acid-based surface additive is sulphuric acid, phosphoric acid, nitric acid, citric acid, or hydrochloric acid.

The present invention further provides a method for converting a light-emitting device into the apparatus as hereinbefore described. Preferably, the light-emitting device is a portable handheld electrical device such as torch light and mobile phone or a non-portable electrical device such as streetlight and wall lamp. The light can be provided by light emitting diode (LED), fluorescent light bulb, and incandescent light bulb. Firstly, the composition as hereinbefore described is prepared, preferably by means of ultrasonication. Preferably, the vibration-energy-containing nanoparticles are added to the organic solvent after the binder during the ultrasonication to ensure formation of a homogeneous mixture. Then, the composition can be deposited onto a surface of the light-emitting device. Subsequently, the composition can be cured at room temperature to form a coating thereonto. Preferably, the deposition is performed by using spraying means such as an atomizer. Prior to the deposition, the surface of the light-emitting device is preferably cleaned to remove impurities such as dust or oil stain, thereby enhancing binding of the composition thereonto. Preferably, the coating has a thickness of about 1 μm to about 10 μm.

Advantageously, the apparatus in the present invention does not require to come into direct contact with the target surface or air containing microbes for disinfection. When in use, the apparatus illuminates the target surface or air thereby transferring the vibration energy of the vibration-energy-containing nanoparticles thereonto via irradiation provided by the light-emitting device. The vibration energy is then transferred to the microbes. In this process, microbial cell walls or cell membranes are induced to vibrate at that frequency. The microbial cell membranes or cell walls have a natural frequency in which upon vibrated at such frequency will cause it to shatter or break. When the frequency transferred to the microbes matches with the natural frequency of the microbial cell membranes or cell walls, such vibration causes them to shatter and break apart, thereby killing the microbes. Based on the aforementioned, the apparatus in the present invention is affordable and convenient to use.

### EXAMPLE

The following non-limiting examples have been carried out to illustrate the preferred embodiments of the present invention.

### Example 1

Some exemplary compositions for forming the coating are tabulated in Tables 1-5.

TABLE 1
First exemplary composition of the present invention.

| Material | Amount (parts by weight) |
|---|---|
| Tetraethoxysilane | 30 |
| Copper sulfate | 1 |
| Alkoxide | 61 |
| Isoprobanol | 8 |

TABLE 2
Second exemplary composition of the present invention.

| Material | Amount (parts by weight) |
|---|---|
| Methylsilane | 15 |
| Sulphuric acid | 1 |
| Methanol | 75 |
| Copper oxide | 9 |

TABLE 3
Third exemplary composition of the present invention.

| Material | Amount (parts by weight) |
|---|---|
| Methylsilane | 8 |
| Dimethyl diethoxysilane | 8 |
| Sulphuric acid | 1 |
| Methanol | 80.5 |
| Copper oxide | 2.5 |

TABLE 4
Fourth exemplary composition of the present invention.

| Material | Amount (parts by weight) |
|---|---|
| Dimethyl diethoxysilane | 10 |
| Tetraethoxysilane | 10 |
| Sulphuric acid | 0.5 |
| Ethanol | 73.5 |
| Copper oxide | 3 |
| Silver oxide | 3 |

TABLE 5
Fifth exemplary composition of the present invention.

| Material | Amount (parts by weight) |
|---|---|
| Dimethysiloxane | 3 |
| Dimethyl diethoxysilane | 7.5 |
| Tetraethoxysilane | 7.5 |
| Sulphuric acid | 0.5 |
| Ethanol | 75.5 |
| Copper oxide | 3 |
| Silver oxide | 3 |

### Example 2

A first experiment was conducted to determine the disinfection capability of the present invention. The composition described in the present invention was deposited onto a surface of a 10 W round LED lamp to form a coating, referred herein as “10 W LED lamp A”. 10 W LED lamp A was compared to a conventional 10 W round LED lamp without the coating, referred herein as “10 W LED lamp B” to determine their disinfection capability. Each of the lamps was positioned at 600 mm on top of an inoculum culture having an initial concentration of 4.9-5.2×10⁴ cfu/ml of Klebsiella pneumoniae. Then, the inoculum cultures were separately exposed to irradiation provided by the lamps for 1 minute, 2 minutes, 5 minutes, 10 minutes, 60 minutes, 3 hours, 6 hours, and 24 hours. Subsequently, the exposed inoculum cultures were incubated at 35° C. for 24-48 hours. Lastly, remaining microbes were determined. The reduction (%) was calculated based on Equation 1. The experiment was repeated using 24 W square LED lamps. The results were tabulated in Table 6.

$$Reduction ( \% ) = \frac{B - A}{B} \times 100 \% \qquad ( Equation 1 )$$

where
A=remaining microbes after irradiation by the LED lamp A (with coating)
B=remaining microbes after irradiation by the LED lamp B (without coating)

TABLE 6
Disinfection capability of 10 W and 24 W LED lamps A and
B at a distance of 600 mm from the inoculum cultures.

| | | | Remaining microbes | Reduction |
|---|---|---|---|---|
| Exposure duration | | Sample | (cfu/ml) | (%) |
| 1 | minute | 10 W LED lamp A | 9.1 × 10³ | 81.8 |
| | | 10 W LED lamp B | 5.0 × 10⁴ | — |
| 2 | minutes | 10 W LED lamp A | 7.2 × 10³ | 85.6 |
| | | 10 W LED lamp B | 5.0 × 10⁴ | — |
| 5 | minutes | 10 W LED lamp A | 4.9 × 10³ | 90.4 |
| | | 10 W LED lamp B | 5.0 × 10⁴ | — |
| 10 | minutes | 10 W LED lamp A | 4.1 × 10³ | 91.8 |
| | | 10 W LED lamp B | 5.0 × 10⁴ | — |
| 60 | minutes | 10 W LED lamp A | 2.5 × 10³ | 95.0 |
| | | 10 W LED lamp B | 5.0 × 10⁴ | — |
| 3 | hours | 10 W LED lamp A | 1.1 × 10³ | 97.8 |
| | | 10 W LED lamp B | 5.0 × 10⁴ | — |
| 6 | hours | 10 W LED lamp A | 8.1 × 10² | 98.4 |
| | | 10 W LED lamp B | 5.1 × 10⁴ | — |
| 24 | hours | 10 W LED lamp A | 3.0 × 10² | 99.4 |
| | | 10 W LED lamp B | 5.1 × 10⁴ | — |
| 1 | minute | 24 W LED lamp A | 4.6 × 10³ | 90.8 |
| | | 24 W LED lamp B | 5.0 × 10⁴ | — |
| 2 | minutes | 24 W LED lamp A | 4.2 × 10³ | 91.6 |
| | | 24 W LED lamp B | 5.0 × 10⁴ | — |
| 5 | minutes | 24 W LED lamp A | 3.1 × 10³ | 93.9 |
| | | 24 W LED lamp B | 5.1 × 10⁴ | — |
| 10 | minutes | 24 W LED lamp A | 1.4 × 10³ | 97.3 |
| | | 24 W LED lamp B | 5.0 × 10⁴ | — |
| 60 | minutes | 24 W LED lamp A | 4.7 × 10² | 99.1 |
| | | 24 W LED lamp B | 4.9 × 10⁴ | — |
| 3 | hours | 24 W LED lamp A | 2.0 × 10² | 99.6 |
| | | 24 W LED lamp B | 5.2 × 10⁴ | — |
| 6 | hours | 24 W LED lamp A | 1.4 × 10² | 99.7 |
| | | 24 W LED lamp B | 5.2 × 10⁴ | — |
| 24 | hours | 24 W LED lamp A | 5.5 × 10¹ | 99.9 |
| | | 24 W LED lamp B | 5.1 × 10⁴ | — |

The microbes in the inoculum cultures were reduced by the irradiation provided by 10 W LED lamp A and 24 W LED lamp A. Additionally, the reduction was higher after a longer exposure. On the contrary, the irradiation provided by 10 W LED lamp B and 24 W LED lamp B did not reduce the number of microbes in the inoculum cultures. The results show that the apparatus in the present invention is capable of providing disinfection capability. In particular, vibration energy of vibration-energy-containing nanoparticles is transferred onto a target surface (the inoculum culture) via irradiation provided by a light-emitting device (LED lamps) for disinfection. It should be noted that the LED lamps used herein do not provides germicidal UV light which by itself is capable of disinfecting microbes.

### Example 3

A second experiment was conducted to determine the disinfection capability of the present invention. The composition described in the present invention was deposited onto a surface of a 18 W LED lamp to form a coating, referred herein as “18 W LED lamp A”. 18 W LED lamp A was compared to a conventional 18 W LED lamp without the coating, referred herein as “18 W LED lamp B” to determine their disinfection capability. Each of the lamps was positioned at 600 mm on top of an inoculum culture having an initial concentration of 9.5-9.6×10⁴ cfu/ml of Klebsiella pneumoniae. Then, the inoculum cultures were separately exposed to irradiation provided by the lamps for 1, 2, 5, 10, 30, and 60 minutes. Subsequently, the exposed inoculum cultures were incubated at 35° C. for 24-48 hours. Lastly, remaining microbes and reduction were determined. The experiment was repeated using 36 W LED lamps. The results were tabulated in Table 7.

TABLE 7
Disinfection capability of 18 W and 36 W LED lamps A and
B at a distance of 600 mm from the inoculum cultures.

| Exposure duration | | Remaining microbes | Reduction |
|---|---|---|---|
| (minutes) | Sample | (cfu/ml) | (%) |
| 1 | 18 W LED lamp A | 8.2 × 10³ | 91.4 |
| | 18 W LED lamp B | 9.5 × 10⁴ | — |
| 2 | 18 W LED lamp A | 7.6 × 10³ | 92.0 |
| | 18 W LED lamp B | 9.5 × 10⁴ | — |
| 5 | 18 W LED lamp A | 6.1 × 10³ | 93.6 |
| | 18 W LED lamp B | 9.5 × 10⁴ | — |
| 10 | 18 W LED lamp A | 4.0 × 10³ | 95.8 |
| | 18 W LED lamp B | 9.5 × 10⁴ | — |
| 30 | 18 W LED lamp A | 1.4 × 10³ | 98.5 |
| | 18 W LED lamp B | 9.5 × 10⁴ | — |
| 60 | 18 W LED lamp A | 7.2 × 10³ | 92.4 |
| | 18 W LED lamp B | 9.5 × 10⁴ | — |
| 1 | 36 W LED lamp A | 8.0 × 10³ | 91.6 |
| | 36 W LED lamp B | 9.5 × 10⁴ | — |
| 2 | 36 W LED lamp A | 7.3 × 10³ | 92.3 |
| | 36 W LED lamp B | 9.5 × 10⁴ | — |
| 5 | 36 W LED lamp A | 4.9 × 10³ | 94.8 |
| | 36 W LED lamp B | 9.5 × 10⁴ | — |
| 10 | 36 W LED lamp A | 2.4 × 10³ | 97.5 |
| | 36 W LED lamp B | 9.5 × 10⁴ | — |
| 30 | 36 W LED lamp A | 9.1 × 10² | 99.0 |
| | 36 W LED lamp B | 9.5 × 10⁴ | — |
| 60 | 36 W LED lamp A | 5.2 × 10² | 99.5 |
| | 36 W LED lamp B | 9.5 × 10⁴ | — |

The microbes in the inoculum cultures were reduced by the irradiation provided by 18 W LED lamp A and 36 W LED lamp A. Additionally, the reduction was higher after a longer exposure. On the contrary, the irradiation provided by 18 W LED lamp B and 36 W LED lamp B did not reduce the number of microbes in the inoculum cultures. The results are in accordance with the findings in the first experiment. It should be noted that the LED lamps used herein do not provides germicidal UV light which by itself is capable of disinfecting microbes.

### Example 4

A third experiment was conducted to determine the disinfection capability of the present invention. The composition described in the present invention was deposited onto a surface of a 36 W LED lamp to form a coating, referred herein as “36 W LED lamp A”. 36 W LED lamp A was compared to a conventional 36 W LED lamp without the coating, referred herein as “36 W LED lamp B” to determine their disinfection capability. Each of the lamps was positioned at 150 mm on top of an inoculum culture having an initial concentration of 2.3-2.6×10⁷ cfu/ml of Klebsiella pneumoniae. Then, the inoculum cultures were separately exposed to irradiation provided by the lamps for 1 minute, 2 minutes, 5 minutes, 10 minutes, 30 minutes, 60 minutes, 3 hours, 6 hours, and 24 hours. Subsequently, the exposed inoculum cultures were incubated at 35° C. for 24-48 hours. Lastly, remaining microbes and reduction were determined. The results were tabulated in Table 8.

TABLE 8
Disinfection capability of 36 W LED lamps A and B
at a distance of 150 mm from the inoculum cultures.

| | | | Remaining microbes | Reduction |
|---|---|---|---|---|
| Exposure duration | | Sample | (cfu/ml) | (%) |
| 1 | minute | 36 W LED lamp A | 2.2 × 10⁶ | 90.8 |
| | | 36 W LED lamp B | 2.4 × 10⁷ | — |
| 2 | minutes | 36 W LED lamp A | 1.7 × 10⁶ | 92.9 |
| | | 36 W LED lamp B | 2.4 × 10⁷ | — |
| 5 | minutes | 36 W LED lamp A | 1.1 × 10⁶ | 95.4 |
| | | 36 W LED lamp B | 2.4 × 10⁷ | — |
| 10 | minutes | 36 W LED lamp A | 5.4 × 10⁵ | 97.8 |
| | | 36 W LED lamp B | 2.4 × 10⁷ | — |
| 30 | minutes | 36 W LED lamp A | 2.2 × 10⁵ | 99.1 |
| | | 36 W LED lamp B | 2.4 × 10⁷ | — |
| 60 | minutes | 36 W LED lamp A | 9.9 × 10⁴ | 99.6 |
| | | 36 W LED lamp B | 2.4 × 10⁷ | — |
| 3 | hours | 36 W LED lamp A | 7.2 × 10⁴ | 99.7 |
| | | 36 W LED lamp B | 2.4 × 10⁷ | — |
| 6 | hours | 36 W LED lamp A | 4.2 × 10⁴ | 99.8 |
| | | 36 W LED lamp B | 2.4 × 10⁷ | — |
| 24 | hours | 36 W LED lamp A | 2.2 × 10⁴ | 99.9 |
| | | 36 W LED lamp B | 2.4 × 10⁷ | — |

The microbes in the inoculum cultures were reduced by the irradiation provided by 36 W LED lamp A. Additionally, the reduction was higher after a longer exposure. On the contrary, the irradiation provided by 36 W LED lamp B did not reduce the number of microbes in the inoculum cultures. The results are in accordance with the findings in the first and second experiments. It should be noted that the LED lamps used herein do not provides germicidal UV light which by itself is capable of disinfecting microbes.

### Example 5

A fourth experiment was conducted to determine the disinfection capability of the present invention. The composition described in the present invention was deposited onto a surface of a 10 W round LED lamp to form a coating, referred herein as “Round LED lamp A”. Round LED lamp A was compared to a conventional 10 W round LED lamp without the coating, referred herein as “Round LED lamp B” to determine their disinfection capability. Each of the lamps was positioned at 600 mm on top of an inoculum culture having an initial concentration of 200-300 cfu/ml of Escherichia coli. Then, the inoculum cultures were separately exposed to irradiation provided by the lamps for 1 minute, 2 minutes, 5 minutes, 10 minutes, and 60 minutes. Subsequently, the exposed inoculum cultures were incubated at 35° C. for 24-48 hours. Lastly, remaining microbes and reduction were determined. The experiment was repeated using Staphylococcus aureus and Klebsiella pneumonia. Then, the experiment was repeated using 24 W square LED lamp, in which the 24 W square LED lamp with the coating is referred herein as “24 W LED lamp A” and the 24 W square LED lamp without the coating is referred herein as “24 W LED lamp B”. The results were tabulated in Tables 9-11 for Escherichia coli, Staphylococcus aureus, and Klebsiella pneumonia, respectively.

TABLE 9
Disinfection capability of LED lamps A and B at a distance of 600
mm from the inoculum cultures containing Escherichia coli.

| | | | Remaining microbes | Reduction |
|---|---|---|---|---|
| Exposure duration | | Sample | (cfu/ml) | (%) |
| 1 | minute | Round LED lamp A | 28 | 87.93 |
| | | Round LED lamp B | 232 | — |
| 2 | minutes | Round LED lamp A | 26 | 88.79 |
| | | Round LED lamp B | 239 | — |
| 5 | minutes | Round LED lamp A | 20 | 91.38 |
| | | Round LED lamp B | 233 | — |
| 10 | minutes | Round LED lamp A | 18 | 92.24 |
| | | Round LED lamp B | 341 | — |
| 60 | minutes | Round LED lamp A | 11 | 95.26 |
| | | Round LED lamp B | 239 | — |
| 1 | minute | Square LED lamp A | 34 | 85.71 |
| | | Square LED lamp B | 238 | — |
| 2 | minutes | Square LED lamp A | 31 | 87.14 |
| | | Square LED lamp B | 241 | — |
| 5 | minutes | Square LED lamp A | 27 | 88.70 |
| | | Square LED lamp B | 239 | — |
| 10 | minutes | Square LED lamp A | 24 | 90.80 |
| | | Square LED lamp B | 240 | — |
| 60 | minutes | Square LED lamp A | 19 | 92.24 |
| | | Square LED lamp B | 245 | — |

TABLE 10
Disinfection capability of LED lamps A and B at a distance of 600
mm from the inoculum cultures containing Staphylococcus aureus.

| | | | Remaining microbes | Reduction |
|---|---|---|---|---|
| Exposure duration | | Sample | (cfu/ml) | (%) |
| 1 | minute | Round LED lamp A | 32 | 88.19 |
| | | Round LED lamp B | 271 | — |
| 2 | minutes | Round LED lamp A | 31 | 88.43 |
| | | Round LED lamp B | 268 | — |
| 5 | minutes | Round LED lamp A | 28 | 89.81 |
| | | Round LED lamp B | 275 | — |
| 10 | minutes | Round LED lamp A | 23 | 91.39 |
| | | Round LED lamp B | 267 | — |
| 60 | minutes | Round LED lamp A | 18 | 93.26 |
| | | Round LED lamp B | 277 | — |
| 1 | minute | Square LED lamp A | 28 | 88.52 |
| | | Square LED lamp B | 244 | — |
| 2 | minutes | Square LED lamp A | 23 | 90.84 |
| | | Square LED lamp B | 251 | — |
| 5 | minutes | Square LED lamp A | 16 | 93.80 |
| | | Square LED lamp B | 258 | — |
| 10 | minutes | Square LED lamp A | 10 | 96.05 |
| | | Square LED lamp B | 253 | — |
| 60 | minutes | Square LED lamp A | 8 | 96.93 |
| | | Square LED lamp B | 261 | — |

TABLE 11
Disinfection capability of LED lamps A and B at a distance of 600
mm from the inoculum cultures containing Klebsiella pneumonia.

| | | | Remaining microbes | Reduction |
|---|---|---|---|---|
| Exposure duration | | Sample | (cfu/ml) | (%) |
| 1 | minute | Round LED lamp A | 31 | 87.45 |
| | | Round LED lamp B | 247 | — |
| 2 | minutes | Round LED lamp A | 30 | 87.76 |
| | | Round LED lamp B | 245 | — |
| 5 | minutes | Round LED lamp A | 25 | 89.75 |
| | | Round LED lamp B | 244 | — |
| 10 | minutes | Round LED lamp A | 21 | 91.60 |
| | | Round LED lamp B | 250 | — |
| 60 | minutes | Round LED lamp A | 16 | 93.52 |
| | | Round LED lamp B | 247 | — |
| 1 | minute | Square LED lamp A | 24 | 90.48 |
| | | Square LED lamp B | 252 | — |
| 2 | minutes | Square LED lamp A | 21 | 91.57 |
| | | Square LED lamp B | 249 | — |
| 5 | minutes | Square LED lamp A | 12 | 95.06 |
| | | Square LED lamp B | 243 | — |
| 10 | minutes | Square LED lamp A | 9 | 96.41 |
| | | Square LED lamp B | 251 | — |
| 60 | minutes | Square LED lamp A | 2 | 99.17 |
| | | Square LED lamp B | 242 | — |

The results show that Escherichia coli, Staphylococcus aureus, and Klebsiella pneumonia in their respective inoculum cultures were reduced by the irradiation provided by Round LED lamp A and Square LED lamp A. On the contrary, the irradiation provided by Round LED lamp B and Square LED lamp B did not reduce the number of microbes in the inoculum cultures. The results are in accordance with the findings in the abovementioned experiments. It should be noted that the LED lamps used herein do not provides germicidal UV light which by itself is capable of disinfecting microbes.

### Example 6

A fifth experiment was conducted to determine the disinfection capability of the present invention. The composition described in the present invention was deposited onto a surface of a 36 W LED lamp (4 feet in length, approximately 1.2 m) to form a coating. The LED lamp was installed in an unsterilized store room. Air sampling was taken at 2 minutes per sample with 100 L/min air flow rate. The post-air sampling was conducted after room had been exposed by irradiation of the LED lamp for 30 minutes, 1 hour, 3 hours, 6 hours, and 24 hours. Tryptic Soy Agar and Malt Extract Agar were used as a sampling media to determine bacteria population and fungi population, respectively. The results were tabulated in Table 12.

TABLE 12
Disinfection capability of 36 W LED lamps
A and B in an unsterilized store room.

| | Initial | | | Post-exposure | |
|---|---|---|---|---|---|
| | population | Exposure | | population | Reduction |
| Parameter | (cfu/m³) | duration | | (cfu/m³) | (%) |
| Total | 275 | 30 | minutes | 205 | 25.45 |
| bacterial | | 1 | hour | 115 | 58.18 |
| count | | 3 | hours | 65 | 76.36 |
| | | 6 | hours | 20 | 92.73 |
| | | 24 | hours | 15 | 94.55 |
| Total fungal | 460 | 30 | minutes | 260 | 43.48 |
| count | | 1 | hour | 120 | 73.91 |
| | | 3 | hours | 55 | 88.04 |
| | | 6 | hours | 55 | 88.04 |
| | | 24 | hours | 50 | 89.13 |

The results show that the bacteria and fungi in the room were reduced by irradiation provided by the LED lamp. The results are in accordance with the findings in the abovementioned experiments. It should be noted that the LED lamps used herein do not provides germicidal UV light which by itself is capable of disinfecting microbes.

One skilled in the art will readily appreciate that the present invention is well adapted to carry out the objects and obtain the ends and advantages mentioned, as well as those inherent therein. The embodiment described herein is not intended as limitations on the scope of the present invention.

1. An apparatus for disinfecting a target surface or air comprising:
(a) a light-emitting device; and
(b) a coating deposited onto a surface of the light-emitting device, wherein the coating is derived from a composition comprising vibration-energy-containing nanoparticles selected from the group consisting of metal-based nanoparticles, rare earth-based nanoparticles, graphene nanoparticles, and any combinations thereof, a binder, an acid-based surface additive, and an organic solvent, whereby the apparatus illuminates the target surface or air upon use thereby transferring the vibration energy of the vibration-energy-containing nanoparticles thereonto so as to disinfect the target surface or air, wherein the vibration-energy-containing nanoparticles vibrate at a frequency of 1 to 1000 kHz to disinfect the target surface or air.

2. The apparatus according to claim 1, wherein the vibration-energy-containing nanoparticles are present at about 0.1% to about 10% by weight of the composition, the binder is present at about 0.1% to about 30% by weight of the composition, the surface additive is present at about 0.1% to about 8% by weight of the composition, and the organic solvent is present at about 75% to about 94% by weight of the composition.

3. The apparatus according to claim 1, wherein the metal-based nanoparticles are derived from metal, metal oxide, metal nitrate, metal sulfate, or any combinations thereof.

4. The apparatus according to claim 1, wherein the rare earth-based nanoparticles are derived from scandium, yttrium, lanthanum, cerium, praseodymium, neodymium, promethium, samarium, europium, gadolinium, terbium, dysprosium, holmium, erbium, thulium, ytterbium, lutetium, or any combinations thereof.

5. The apparatus according to claim 1, wherein the binder is a silane.

6. The apparatus according to claim 1, wherein the acid-based surface additive is sulphuric acid, phosphoric acid, nitric acid, citric acid, or hydrochloric acid.

7. The apparatus according to claim 1, wherein the organic solvent is an alcohol, a silicone oil, or a combination thereof.

8. The apparatus according to claim 1, wherein the coating has a thickness of about 1 μm to about 10 μm.
